(19) United States
(12) Patent Application Publication
(10) Pub. No.: US 2019/0262184 A1
(43) Pub. Date: Aug. 29, 2019

(54) Therapy Tape

(71) Applicant: Wesley G. Wilson, (US)

(72) Inventor: Wesley G. Wilson, Scottsdale, AZ (US)

(21) Appl. No.: 16/040,087

(22) Filed: Jul. 19, 2018

Related U.S. Application Data
(60) Provisional application No. 62/635,534, filed on Feb. 26, 2018.

(51) Int. Cl. A61F 13/02; A61L 15/58

(52) U.S. Cl. CPC A61F 13/024; A61F 13/0253; A61L 15/58

(57) ABSTRACT

An apparatus is disclosed. The apparatus includes a first strip of flexible material, a second strip of flexible material, and a stretchable strip. The first strip of flexible material has a length and a width. The second strip of flexible material is disposed on the first strip of flexible material. The stretchable strip is disposed on the second strip of flexible material. The stretchable strip protrudes from a plane of the first side of the second strip of flexible material. The stretchable strip of material extends predominantly along the length of the second strip of flexible material. The stretchable strip of material has a width, in at least one cross-section, that is less than the width of the first strip of flexible material.

### CROSS-REFERENCE TO RELATED APPLICATIONS

This application claims the benefit of U.S. Provisional Application No. 62/635,534, filed on Feb. 26, 2018, which is incorporated by reference herein in its entirety.

### FIELD

This invention relates to medical tapes and more particularly relates to wound therapy tapes and treatments.

### BACKGROUND

Medical tapes are used to address injury, wounds, incisions, and provide functionality in medical procedures and therapies. Tapes can be made sterile to reduce the chance of infection and may provide an easy-to-use product with relatively low cost and complexity.

### SUMMARY

An apparatus is disclosed. The apparatus includes a first strip of flexible material, a second strip of flexible material, and a stretchable strip. The first strip of flexible material has a length and a width. The second strip of flexible material is disposed on the first strip of flexible material. The stretchable strip is disposed on the second strip of flexible material. The stretchable strip protrudes from a plane of the first side of the second strip of flexible material. The stretchable strip of material extends predominantly along the length of the second strip of flexible material. The stretchable strip of material has a width, in at least one cross-section, that is less than the width of the first strip of flexible material.

A system is also described. The system includes a therapy tape and a removable backing strip. The therapy tape includes a first strip of flexible material, a second strip of flexible material, and a stretchable strip. The first strip of flexible material has a length and a width. The second strip of flexible material is disposed on the first strip of flexible material. The stretchable strip is disposed on the second strip of flexible material. The stretchable strip protrudes from a plane of the first side of the second strip of flexible material. The stretchable strip of material extends predominantly along the length of the second strip of flexible material. The stretchable strip of material has a width, in at least one cross-section, that is less than the width of the first strip of flexible material. The backing strip is applied to the first and second strips of flexible material of the therapy tape. The backing strip has a backing length and a backing width. The removable backing strip or release paper is also over the stretchable strip, as well.

A method is also described. The method includes forming a strip of flexible material to have a length and a width. The strip of flexible material has a two-way stretch parallel to the length of the strip of flexible material. The method further includes applying adhesive to at least a portion of a first side of the strip of flexible material. The method further includes disposing a stretchable strip on the first side of the strip of flexible material. The stretchable strip protrudes from a plane of the fist side of the strip of flexible material. The stretchable strip also extends predominantly along the length of the strip of flexible material. The stretchable strip has a width, in at least one cross-section, that is less than the width of the strip of flexible material.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1A is a schematic diagram illustrating one embodiment of a therapy tape in accordance with one embodiment of the present invention.

FIG. 1B is a schematic diagram illustrating another embodiment of a therapy tape.

FIG. 1C is a sectional view of the therapy tape of FIG. 1B.

FIG. 1D is a schematic diagram illustrating another embodiment of a therapy tape.

FIG. 2 is a perspective view of another embodiment of the therapy tape of FIG. 1A with a perforated backing.

FIG. 3A is a perspective view of another embodiment of the therapy tape of FIG. 1A with an overlap backing.

FIG. 3B is a perspective view of another embodiment of the therapy tape of FIG. 1A with and overlap backing having a central portion.

FIG. 4A is a schematic diagram of one embodiment of a strip of flexible material for a therapy tape.

FIG. 4B is a schematic diagram of another embodiment of a strip of flexible material for a therapy tape.

FIG. 4C is a schematic diagram of a further embodiment of a strip of flexible material for a therapy tape.

FIG. 5 is a flow diagram illustrating one embodiment of a method of using a therapy tape.

FIG. 6 is a flow diagram illustrating one embodiment of a method of making a therapy tape.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

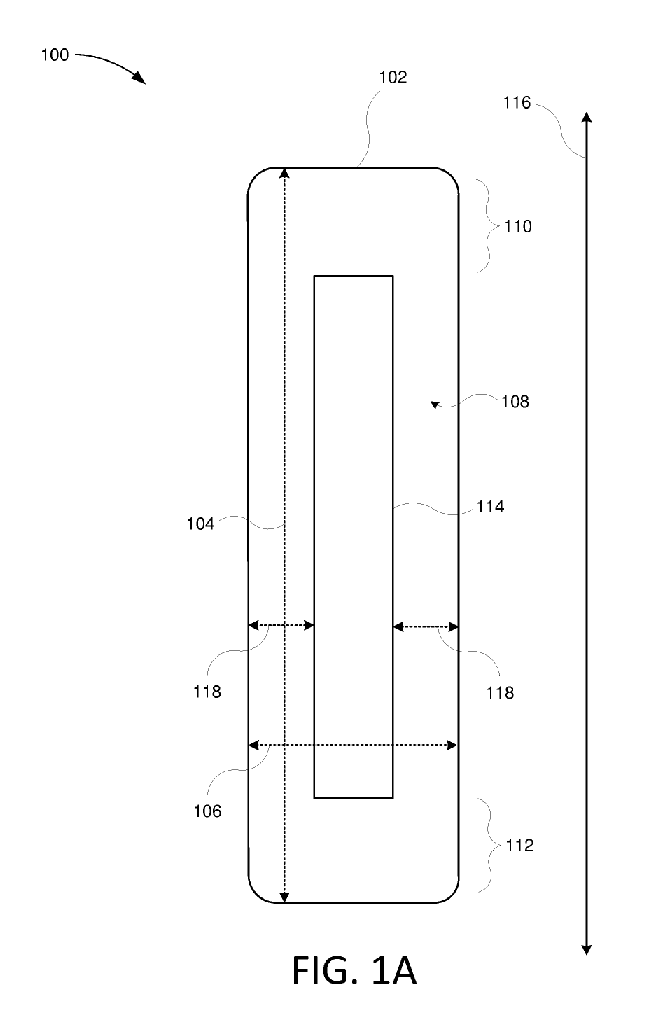

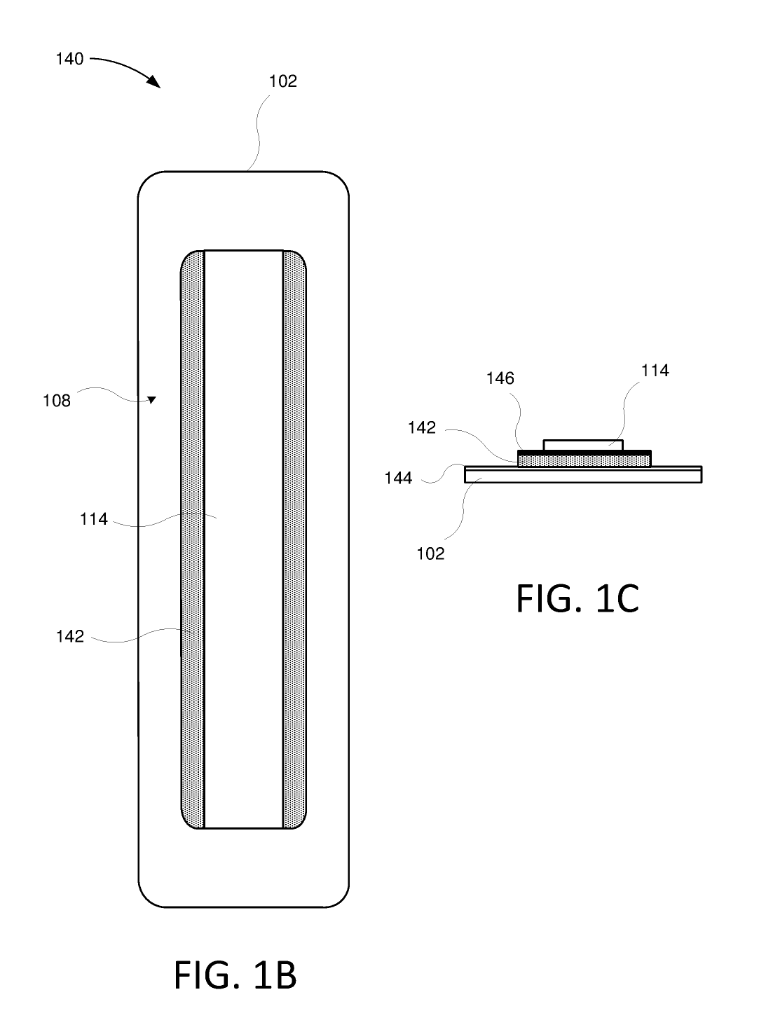

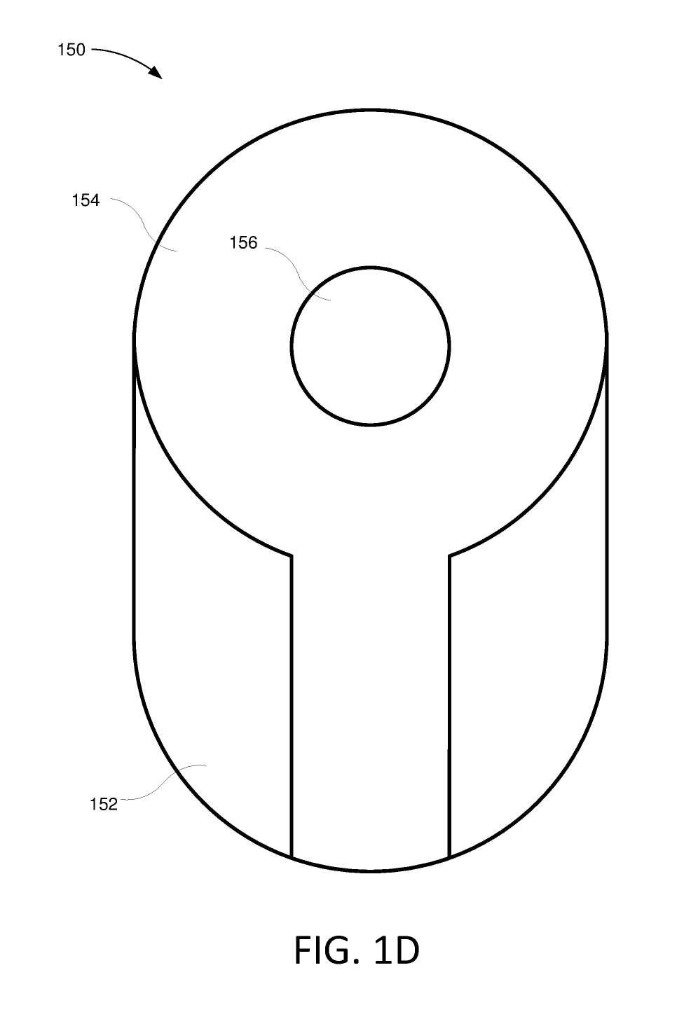

FIG. 1A is a schematic diagram illustrating one embodiment of a therapy tape 100 in accordance with one embodiment of the present invention. In the illustrated embodiment, the therapy tape 100 includes a strip of flexible material 102. In some embodiments, the strip of flexible material 102 is a flat, elongated, tape or ribbon-like structure having a planar geometry. In other embodiments, the strip of flexible material 102 may have a different geometry, including any variety of shape contoured or congruent with various parts of a user's body where the therapy tape 100 might be used. In some embodiments, at least a portion of the strip of flexible material 102 includes a fabric. The fabric may be a woven or non-woven fabric. In some embodiments, the fabric has a stretch pattern. The stretch of the strip of flexible material 102 is discussed in more detail below.

The illustrated embodiment of the strip of flexible material 102 has a length 104 which is the greater or major dimension or geometry of the therapy tape 100. The therapy tape 100 also has a width 106. In some embodiments, the width 106 is perpendicular to the length. In the illustrated embodiment, the length 104 is greater or longer than the width 106. In some embodiments, the length 104 extends up to approximately 40 centimeters. In one example, the flexible material 102 may have dimensions of about 40 cm long by about 5 cm wide. In another embodiment, the flexible material 102 may have dimensions of about 18 cm long by about 5 cm wide. In another embodiment, the flexible material 102 may have dimensions of about 7.5 cm long by about 2.5 cm wide. However, embodiments of the flexible material may have any dimension of length between about 5-50 cm and width between about 1-10 cm, and in certain situations the dimensions may be greater or smaller than these example dimensions.

In the illustrated embodiment, the strip of flexible material 102 includes a first side 108. In some embodiments, the first side 108 is part of or formed on the strip of flexible material 102. The first side 108 may include one or more treatments or structures to facilitate application of the therapy tape 100 to a surface. For example, the first side 108 may have an adhesive disposed on all of or at least a portion of the first side 108. In some embodiments, the adhesive is resistant to wound or incision adhesion. In some embodiments, the surface to which the therapy tape 100 is applied is an area of skin on a human or animal. In some embodiments, the therapy tape 100 is applied to an incision site of a patient.

In some embodiments, the adhesive is hypoallergenic, breathable, chemical-resistant, or otherwise facilitates or benefits interface with human or animal skin. In some embodiments, the adhesive is applied uniformly over a portion of the first side 108. In other embodiments, the adhesive is applied non-uniformly over a portion of the first side 108.

In the illustrated embodiment, the strip of flexible material 102 also includes a first attachment region 110 formed on the first side 108 at a first end of the length 104 of the strip of flexible material 102. In some embodiments, the strip of flexible material 102 also includes a second attachment region 112 formed on the first side 108 at a second end of the strip of flexible material 102 opposite the first end. In some embodiments, the first attachment region 110 and the second attachment region 112 are identical in one or more aspects such as size, shape, or functional characteristics. In other embodiments, the first attachment region 110 and the second attachment region 112 are different from one another.

In some embodiments, at least one of the first attachment region 110 and the second attachment region 112 has relatively lower elasticity relative to one or more other portions of the strip of flexible material 102. For example, one or both of the first attachment region 110 and the second attachment region 112 may have limited stretch in one or both of the length 104 and the width 106. In some embodiments, one or both of the first attachment region 110 and the second attachment region 112 include one or more materials incorporated into the strip of flexible material 102 to reduce the stretch in one or both of the length 104 and the width 106. In some embodiments, the stretch at one or both of the first attachment region 110 and the second attachment region 112 is restricted by a treatment, coating, or other surface structure disposed at the one or both of the first attachment region 110 and the second attachment region 112. Alternatively, the first attachment region 110 and the second attachment region 112 may have the same flexible properties as the rest of the therapy tape 100, and the amount of stretch applied to the portion between the first and second attachment regions 110 and 112 may be different than at the first and second attachment regions 110 and 112 due to the application process used by a physician or patient in applying the therapy tape 100. For example, someone applying the therapy tape 100 may remove a central portion of the backing to stretch the portion between the first and second attachment regions 110 and 112, and subsequently remove the backing at the first and second attachment regions 110 and 112 to apply those regions without stretch (or with a different amount of stretch) to their respective application locations. The backing may be made of paper or plastic.

In the illustrated embodiment, the therapy tape 100 includes a stretchable strip 114. Other embodiments may include more than one stretchable strip 114. In some embodiments, the stretchable strip 114 is formed on the first side 108 of the strip of flexible material 102. In some embodiments, the stretchable strip 114 is formed along the length 104 and protrudes from the first side 108. In some embodiments, the stretchable strip 114 is at least partially made of silicone. In some embodiments, the stretchable strip 114 is a bead or other raised structure formed of silicone or similar material or combination of materials. In some embodiments, the stretchable strip 114 includes active agents to prevent infection, reduce irritation, reduce pain, encourage healing, reduce scarring, or the like. The stretchable strip 114 may be formed before or after an adhesive or other agent is applied to the first side 108 of the strip of flexible material 102.

In some embodiments, the stretchable strip 114 extends predominantly along the length of the strip of flexible material. The stretchable strip 114 may extend the entire length of the flexible material 102 or a partial length of the flexible materials 102. The term “predominant” means having a greater dimension in the lengthwise direction than in other directions. The term “predominant” also may mean that the ratio of the length of the stretchable strip 114 to the length of the strip of flexible material 102 is greater than the ratio of the ratio of the width of the stretchable strip 114 to the width of the strip of flexible material 102.

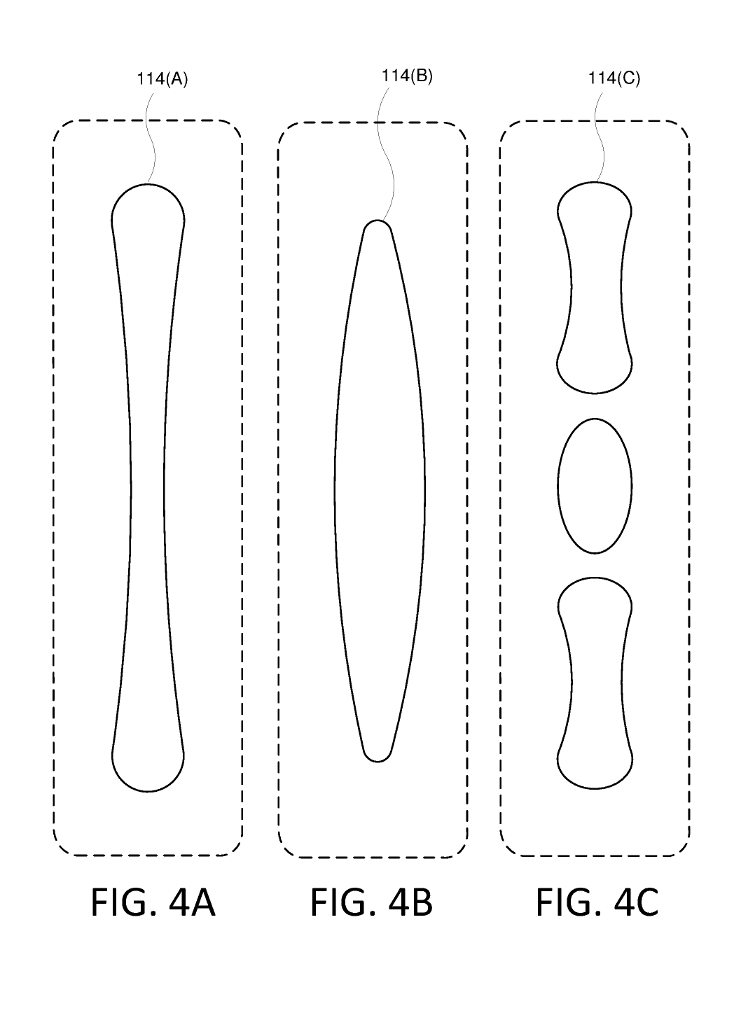

The illustrated embodiment of the stretchable strip 114 is rectangular. In other embodiments, the stretchable strip 114 is non-rectangular. For example, the stretchable strip 114 may be oval, elliptical, tear-drop, tapered, segmented, or the like. In some embodiments, the stretchable strip 114 has rounded, squared, angled, tapered, or other edge treatments. Additionally, the stretchable strip 114 may be uniform, non-uniform, continuous, non-continuous, symmetrical, and non-symmetrical. Embodiments of the stretchable strip 114 are further described below with respect to FIGS. 4A-C.

In the illustrated of FIG. 1A, the stretchable strip 114 extends from the first attachment region 110 to the second attachment region 112. In some embodiments, the stretchable strip 114 may be positioned closer to one of the first attachment region 110 or the second attachment region 112 than the other. In the illustrated embodiment, the stretchable strip 114 is positioned in a middle of the strip of flexible material 102. In other embodiments, the stretchable strip 114 is positioned nearer one side or another in the width 106 of the strip of flexible material 102.

In some embodiments, the stretchable strip 114 defines one or more margin regions 118. In the illustrated embodiment, the stretchable strip 114 is positioned in the middle of the strip of flexible material 102. With the stretchable strip 114 positioned in the middle of the strip of flexible material 102, the stretchable strip 114 defines two symmetrical margin regions 118. In some embodiments, each of the margin regions 118 are equal in width, along the width 106, to a width of the stretchable strip 114. In some embodiments, the margin regions 118 are identical. In other embodiments, the margin regions 118 are not identical. For example, the margin regions 118 may not be identical because the stretchable strip 114 may be positioned nearer one side of the strip of flexible material 102 than another, the stretchable strip 114 may have a non-symmetrical geometry, the strip of flexible material 102 may have a non-symmetrical geometry or a non-uniform geometry, or the like.

In the illustrated embodiment, the strip of flexible material 102 exhibits a two-way stretch 116 (approximately along a single dimension such as the length) which is aligned with the length of the strip of flexible material 102. In some embodiments, the two-way stretch 116 of the strip of flexible material 102 facilitates application of the therapy tape 100 to apply pressure to an application site by pressing the stretchable strip 114 against the application site. In some embodiments, the two-way stretch 118 is at least a partial function of one or more materials of the strip of flexible material 102. In other embodiments, the two-way stretch 118 is at least a partial function of the stretchable strip 114. In some embodiments, the two-way stretch 118 is a function resulting from an effect of one or more components of the therapy tape 100.

In some embodiments, the strip of flexible material 102 of the therapy tape 100 exhibits limited, to substantially no, stretch parallel to the width 106. The limited stretch in the width 106 may facilitate reduced stress at an incision which, absent the therapy tape 100 or if the width 106 were allowed to stretch, would open or stretch the incision and hinder healing and scar reduction or introduce damaging or uncomfortable shear forces at the first side 108.

FIG. 1B is a schematic diagram illustrating another embodiment of a therapy tape 140. The depicted therapy tape 140 may be similar in many ways to the therapy tape 100 of FIG. 1A. However, the illustrated therapy tape 140 includes multiple fabric layers 102 and 142, instead of a single fabric layer 102. By using a second fabric layer 142 on top of the first fabric layer 102, the stretchable strip 114 may be applied to only the second fabric layer 142. This approach may allow fabrication of a smaller or more detailed stretchable strip 114, rather than application of the stretchable strip 114 across the length of the first fabric layer 102. Although a single strip of the second fabric 142 and stretchable strip 114 are shown, other embodiments may incorporate multiple discrete segments of the second fabric layer 142. In other embodiments, the stretchable strip 114 may be applied to both the first and second fabric layers 142 in either continuous or discrete applications. Additionally, in some embodiments the length of the stretchable strip 114 may exceed the length of the second fabric layer 142, in one or both directions. In further embodiments, an adhesive may be applied to the exposed surface of the stretchable strip 114.

FIG. 1C is a sectional view of the therapy tape of FIG. 1B. This perspective illustrates a plurality of layers combined to form the therapy tape. Specifically, the depicted sectional view illustrates the first fabric layer 102, a first adhesive 144 applied to part or all of the first fabric layer 102, and the second fabric layer 142 adhered to the first fabric layer 102. In another embodiment, the first adhesive 144 may be applied to the back side of the second fabric layer 142, instead of to the front side of the first fabric layer 102, although the end result is the same to have the first adhesive 144 fastening the two fabric layers 102 and 142 together. A second adhesive 146 may be applied to the top side of the second fabric layer 146 in order to adhere (or increase adherence of) the stretchable strip 114 to the second fabric layer 142. Alternatively, in some embodiments the stretchable strip 114 is made up of a material that does not require the second adhesive 146 in order to adhere to the second fabric layer 142.

When the first and second fabric layers 102 and 142 are adhered to one another, the fabrics are aligned so that the direction of stretch is the same or approximately the same. For example, both the first and second fabric layers 102 and 140 may be aligned to allow stretch of the therapy tape 140 lengthwise, without facilitating stretch across the width of the therapy tape 140.

FIG. 1D is a schematic diagram illustrating another embodiment of a therapy tape 150. The depicted embodiment may incorporate many of the features described herein, even though the shape of the materials differs to accommodate a specific purpose. In one example, the therapy tape 150 has a shape to facilitate use of the therapy tape on or near the breast area of a patient. In the illustrated embodiment, the first fabric layer 152 underlies a second fabric layer 154. The first and second fabric layers 152 and 154 are designed to stretch in one direction (e.g., vertically as shown) and not stretch in the other direction (e.g., horizontally as shown). A stretchable strip, or material, may be applied to part or all of the second fabric layer 154. Additionally, a backing or release material may be applied to the stretchable strip and/or areas of the first and second fabric layer 152 and 154. Both the first and second fabric layers 152 and 154 include a hole to allow passage of part of the patient's body, such as a nipple, through the therapy tape 150. Although not shown specifically, the portion of the therapy tape 150 that is stretchable (or to be stretched during application) may vary depending on the location of the incision or scare to which the therapy tape 150 is applied. In a specific embodiment, the circular portion and a distal end of the second fabric layer 152 are not stretched, while an intermediate portion of the straight segment of the second fabric layer 152 is stretched during application to the patient. Other embodiments may be designed or utilized to stretch other parts of the therapy tape 150.

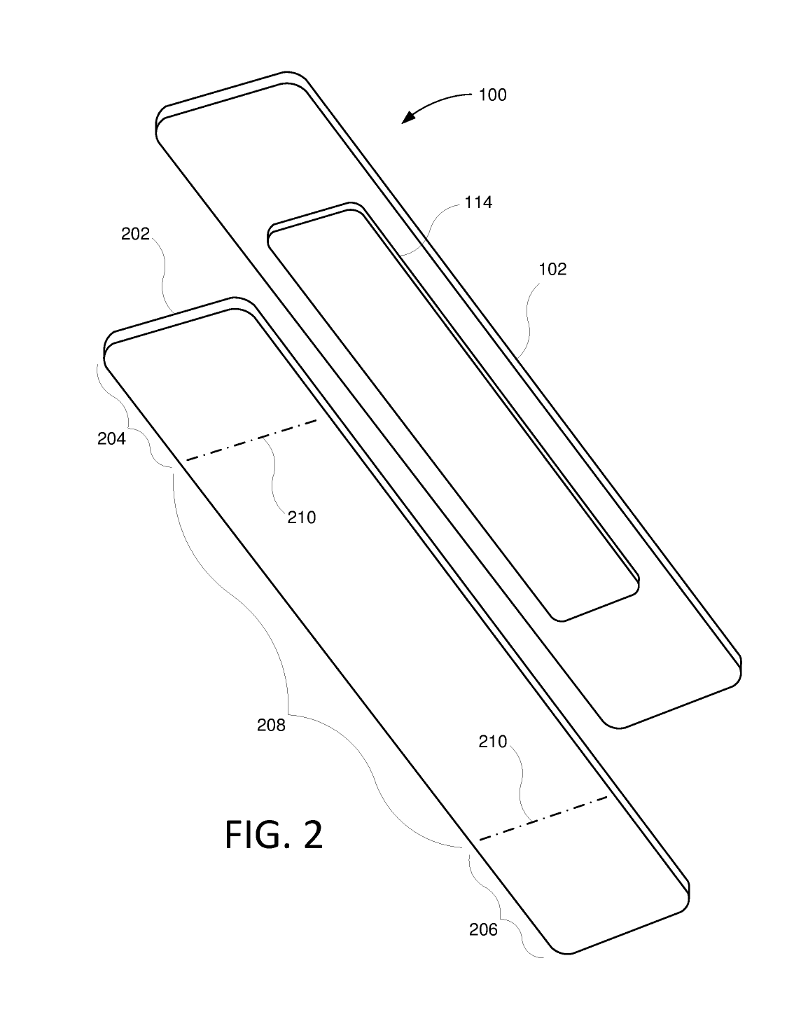

FIG. 2 is a perspective view of another embodiment of the therapy tape 100 of FIG. 1A with a perforated backing 202. Other embodiments may utilize non-perforated markings to indicate suggested locations to tear or separate the backing 202. Accordingly, references herein to perforations should also be understood to refer to non-perforated markings, where applicable. Of course, other embodiments may have a non-perforated and non-marked backing. In the illustrated embodiment, the perforated backing 202 is shown separated from the therapy tape 100 for illustrative purposes. In the illustrated embodiment, the perforated backing 202 includes a first perforated end 204, a second perforated end 206, and a primary backing portion 208.

In some embodiments, the first perforated end 204 and the second perforated end 206 are portions of the perforated backing 202 defined at each end of the perforated backing 202 by perforations 210. In some embodiments, the first perforated end 204 and the second perforated end 206 are identical. In other embodiments, the first perforated end 204 and the second perforated end 206 are different from one another. In some embodiments, the first perforated end 204 and the second perforated end 206 correspond, in at least one of position and geometry, with the first attachment region 110 and the second attachment region 112 as shown and described in FIG. 1A.

In some embodiments, the first perforated end 204 and the second perforated end 206 may be maintained in place on the strip of flexible material 102 of the therapy tape 100 while the primary backing portion 208 is removed. In some embodiments, the first perforated end 204 and the second perforated end 206 reduce stretching of the corresponding first attachment region 110 and the second attachment region 112. This serves to reduce skin stress to reduce blistering, discomfort, and scarring, and improve healing time.

In the illustrated embodiment, the perforations 210 are linear. In other embodiments, one or more of the perforations 210 are curvilinear and/or non-linear. In some embodiments, the perforations 210 are positioned to fully expose the stretchable strip 114 when the primary backing portion 208 is removed and the first perforated end 204 and the second perforated end 206 remain in place on the strip of flexible material 102.

In some embodiments, the geometry of the perforated backing 202 approximately matches the geometry of the strip of flexible material 102. In other embodiments, the geometry of the perforated backing 202 is larger, smaller, or different from the geometry of the strip of flexible material 102. For example, the perforated backing 202 may be skeletonized in some places to save material and weight, extended in some place to provide grip locations to facilitate manipulation of the strip of flexible material 102 and/or perforated backing 202, match adhesive patterns, the strip of flexible material, or other portions or structures of the therapy tape 100.

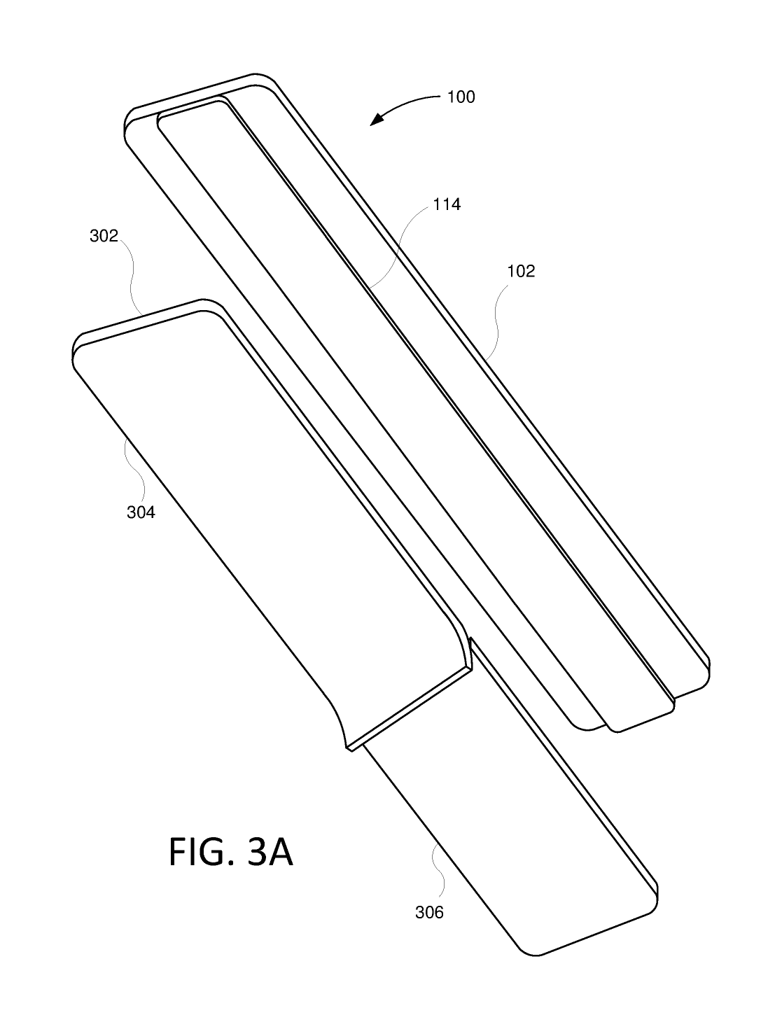

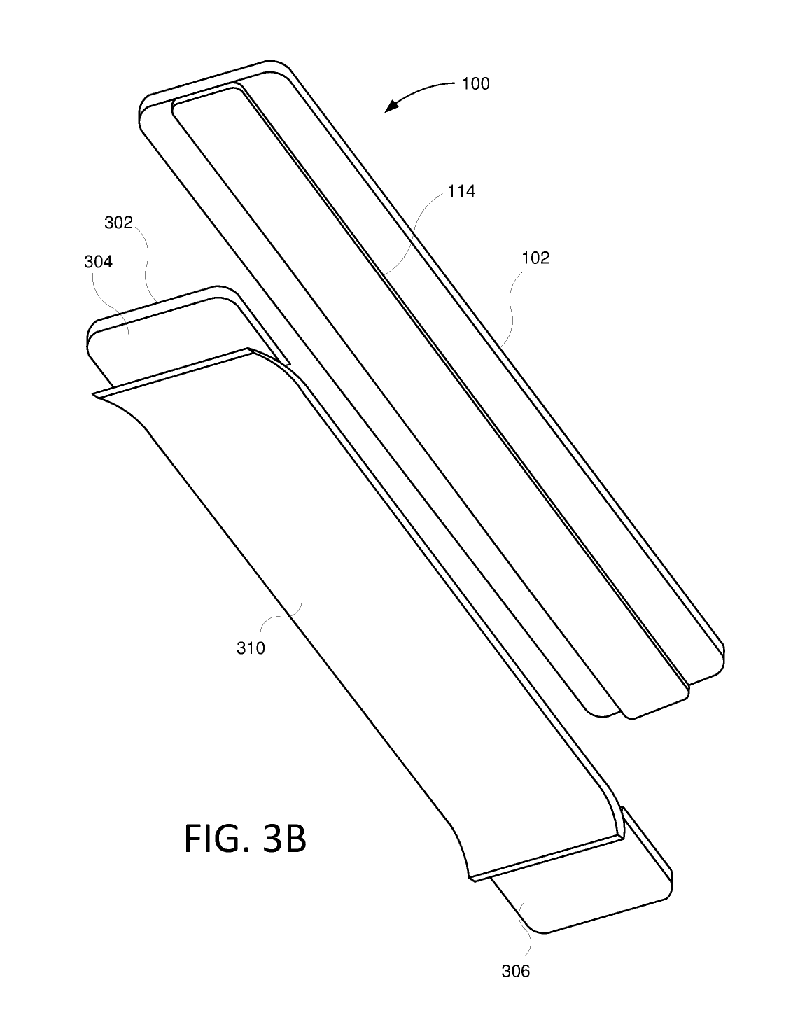

FIG. 3A is a perspective view of another embodiment of the therapy tape 100 of FIG. 1A with an overlap backing 302. In the illustrated embodiment, the overlap backing 302 is shown separated from the therapy tape 100 for illustrative purposes. In the illustrated embodiment, the overlap backing 202 includes a first overlap portion 304 and a second overlap portion 306.

In some embodiments, the first overlap portion 304 and the second overlap portion 306 are portions of the overlap backing 302 sized to overlap one another. In some embodiments, the first overlap portion 304 and the second overlap portion 306 are identical. In other embodiments, the first overlap portion 304 and the second overlap portion 306 are different from one another. In some embodiments, the first overlap portion 304 and the second overlap portion 306 overlap one another at approximately a center of the overlap backing 302 corresponding to a center of the therapy tape 100. In some embodiments, the overlap backing 302 corresponds, in at least one of position and geometry, with the first attachment region 110 and the second attachment region 112 as shown and described in FIG. 1A.

In some embodiments, at least one of the first overlap portion 304 and the second overlap portion 306 includes a portion which may be maintained in place on the strip of flexible material 102 of the therapy tape 100 while the remaining portion of the at least one of the first overlap portion 304 and the second overlap portion 306 is peeled back or otherwise removed. In some embodiments, the first overlap portion 304 and the second overlap portion 306 reduce stretching of the corresponding first attachment region 110 and the second attachment region 112 during application of the therapy tape 100 to an application site. This may serve to reduce skin stress to reduce blistering, discomfort, and scarring, and improve healing time.

In the illustrated embodiment, the thresholds between the first overlap portion 304 and the second overlap portion 306 are linear. In other embodiments, one or more of the thresholds are curvilinear and/or non-linear. In some embodiments, the first overlap portion 304 and the second overlap portion 306 are positioned to at least partially expose the strip of flexible material during application of the therapy tape 100.

In some embodiments, the geometry of the overlap backing 302 approximately matches the geometry of the strip of flexible material 102. In other embodiments, the geometry of the overlap backing 302 is larger, smaller, or different from the geometry of the strip of flexible material 102. For example, the overlap backing 302 may be skeletonized in some places to same material and weight, extended in some place to provide grip locations to facilitate manipulation of the strip of flexible material 102 and/or the overlap backing 202, match adhesive patterns, the stretchable strip 114, or other portions or structures of the therapy tape 100.

In the illustrated embodiment, the stretchable strip 114 extends the full length of the strip of flexible material 102. In some embodiments, the stretchable strip 114 does not fully extend to at least one end of the strip of flexible material 102. In some situations, extension of the stretchable strip 114 to the ends of the strip of flexible material 102 may reduce complexity in use and/or manufacturing.

FIG. 3B is a perspective view of another embodiment of the therapy tape 100 of FIG. 1A with and overlap backing having a central portion 310. In the illustrated embodiment, the length and geometry of the central portion 310 of the removable backing is designed for removal separate from end portions of the removable backing, which allows stretching of the corresponding central portion of the therapy tape 100, without allowing the ends of the therapy tape 150 to stretch.

In other embodiments, other types of backing or release materials may be used. For example, the backing may or may not overlap, or the backing may or may not be perforated, or the backing may or may not be cut and aligned to abut with each other. The various types of backing strips shown and described may be implemented with the various shapes, sizes, lengths, and/or geometries shown and described herein. In general, any component described in a particular embodiment may be implemented or combined with any other feature of another embodiment, unless otherwise noted or rendered impossible by the corresponding configurations.

FIG. 4A is a schematic diagram of one embodiment of a stretchable strip 114(A) for a therapy tape 100. In the illustrated embodiment, the stretchable strip 114(A) has a geometry with a centrally-narrowed or hourglass shape with rounded ends. In some embodiments, the stretchable strip 114(A) may include variations in thickness or surface textures along the length and/or width of the strip of flexible material 114(A). In some embodiments, the stretchable strip 114(A) is 0.9 mm in thickness. In one example, the stretchable strip 114(A) has a thickness between about 0.3 mm to about 0.6 mm. In another embodiment, the stretchable strip 114(A) has a thickness between about 0.2 mm to about 0.6 mm. However, embodiments of the stretchable strip 114(A) may have any dimension of a thickness between about 0.1 mm to about 0.8 mm, and in certain situations the dimensions may be greater or smaller than these example dimensions. Also, the flexible material 102 and 142 may have various thicknesses. In one embodiment, the thickness is about 0.5 mm.

FIG. 4B is a schematic diagram of another embodiment of a stretchable strip 114(B) for a therapy tape 100. In the illustrated embodiment, the strip of stretchable material 114(B) has a geometry with tapered ends and a centrally enlarged shape.

FIG. 4C is a schematic diagram of a further embodiment of a stretchable strip 114(C) for a therapy tape 100. In the illustrated embodiment, the stretchable strip 114(C) is segmented. The outer portions of the stretchable strip 114(C) have geometries which may be similar to that of the stretchable strip 114(A) of FIG. 4A. In the illustrated embodiment, the middle portion of the stretchable strip 114(C) includes an oval geometry. In some embodiments, the different segments of the stretchable strip 114(C) are identical or non-identical. Variations of thicknesses and surface structures and features may also be included.

While the embodiments described above in relation to the various figures describe specific shapes and variations, other embodiments include a wide range of different shapes, thicknesses, treatments, surfaces, and the like. In some embodiments, the geometry of the strip of flexible material is selected based on a nature of the treatment site. For example, the geometry of the strip of flexible material may be selected to match an incision having a corresponding structure or geometry, a type of surgery or wound, a portion of the patient to which the therapy tape 100 is to be applied, and similar considerations.

In some embodiments, a strip of flexible material or other structure or characteristic of the therapy tape 100 may be custom designed for an individual application. For example, following a surgery or wound occurrence, a therapy tape 100 may be designed to fit and treat the individual application. This may include custom fitting for the strip of flexible material 102, the stretchable strip, the adhesive, or other components, aspects, or characteristics of the therapy tape 100 to match the individual.

In some embodiments, the strip of flexible material may have at least partial manipulability to facilitate changes to the shape, features, and/or size of the strip of flexible material. For example, the strip of flexible material may include breakaway portions which may be removed to change the shape, thickness, or the like of the strip of flexible material. In some embodiments, the therapy tape 100, as a whole, may be subdivided to fit the therapy tape 100 to an individual application.

In some embodiments, the therapy tape 100 may be individually packaged. In other embodiments, the therapy tape 100 may be a bulk rolled or continuous strip good. Other manners of manufacturing and organizing the therapy tape 100 are contemplated.

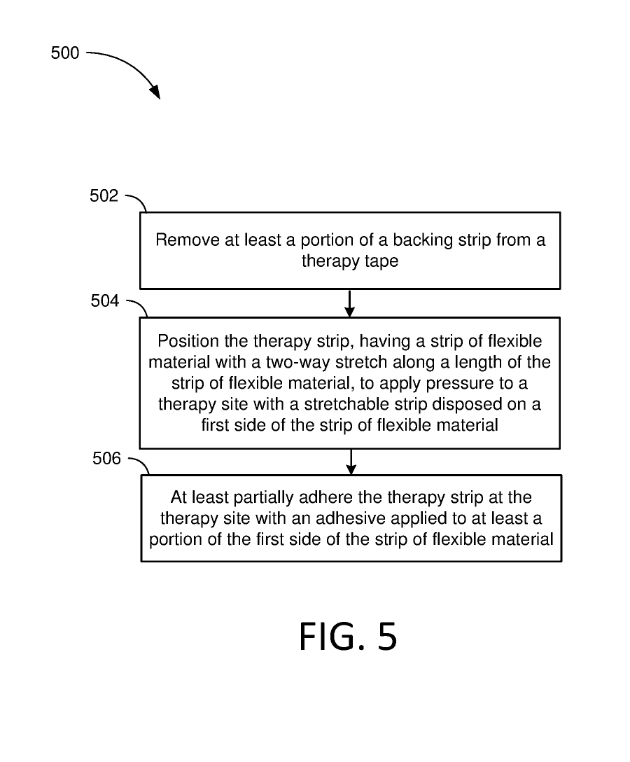

FIG. 5 is a flow diagram illustrating one embodiment of a method 500 of using a therapy tape 100. The illustrated embodiment of the method 500 includes, at block 502, removing at least a portion of a backing strip from a therapy tape. In some embodiments, the backing strip includes one or more portions which remain on the therapy tape. As described above, the backing strip may include perforations for removing or tearing away a portion of the backing strip with portions of the backing strip left in place to facilitate positioning the therapy tape.

At block 504, the method 500 includes positioning the therapy tape, having a strip of flexible material with a two-way stretch along a length of the strip of flexible material, to apply pressure to a therapy site with a stretchable strip disposed on a first side of the strip of flexible material.

At block 506, the method 500 includes at least partially adhering the therapy tape at a therapy site with an adhesive applied to at least a portion of the first side of the strip of flexible material. In some embodiments, the stretchable strip protrudes from a plane of the first side of the strip of flexible material. In some embodiments, the stretchable strip extends predominantly along the length of the strip of flexible material. In some embodiments, the stretchable strip has a width, in at least one cross-section, that is less than the width of the strip of flexible material.

The method 500 may further include remove any remaining portions of the backing strip from the therapy tape. As described above, one or more remaining portions of the backing strip may be left in place to prevent stretching of one or more portions of the strip of flexible material during application of the therapy tape to prevent blistering or other damage or irritation due to excessive forces applied by the two-way elastic characteristic of the therapy tape.

In some embodiments, the therapy tape is fully secured by removing any the remaining portions of the backing strip once the therapy strip is at least partially adhered to the therapy site.

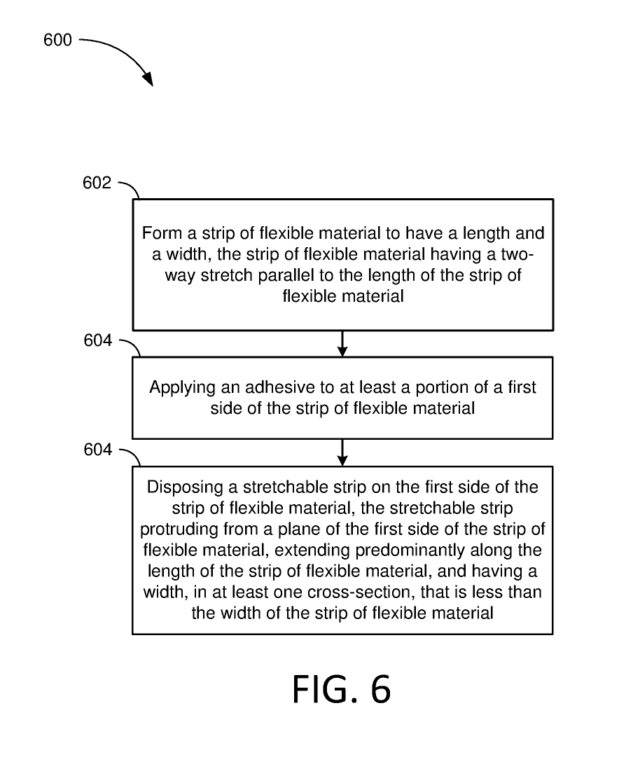

FIG. 6 is a flow diagram illustrating one embodiment of a method 600 of making a therapy tape 100. At block 602, the method 600 includes forming a strip of flexible material to have a length and a width. The strip of flexible material has a two-way stretch parallel to the length of the strip of flexible material.

At block 604, the method 600 includes applying an adhesive to at least a portion of a first side of the strip of flexible material.

At block 606, the method 600 includes disposing a stretchable strip on the first side of the strip of flexible material. The stretchable strip protrudes from a plane of the first side of the strip of flexible material. The stretchable strip extends predominantly along the length of the strip of flexible material. The stretchable strip has a width, in at least on cross-section, that is less than the width of the strip of flexible material.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus comprising:
a first strip of flexible material having a length and a width;
a second strip of flexible material disposed on the first strip of flexible material; and
a stretchable strip disposed on the second strip of flexible material, wherein the stretchable strip:
protrudes from a plane of the first side of the second strip of flexible material;
extends predominantly along the length of the second strip of flexible material; and
has a width, in at least one cross-section, that is less than the width of the first strip of flexible material.

2. The apparatus of claim 1, wherein the first and second strips of flexible material comprise a fabric.

3. The apparatus of claim 1, wherein the stretchable strip comprises a silicone structure.

4. The apparatus of claim 1, wherein the first and second strips of flexible material comprise a two-way stretch parallel to the length of the strip of flexible material.

5. The apparatus of claim 1, wherein at least one end of the first strip of flexible material comprises an attachment region having a lower elasticity than another portion of the first strip of flexible material.

6. The apparatus of claim 1, wherein the stretchable strip extends along the length of the second strip of flexible material without extending to an edge of the first strip of flexible material.

7. The apparatus of claim 1, wherein the first strip of flexible material comprises margin regions on the first side extending along the length of the first strip of flexible material on opposite sides of the stretchable strip, wherein the stretchable strip is disposed along a middle of the first strip of flexible material.

8. The apparatus of claim 1, wherein the stretchable strip is uniform in geometry along a length of the stretchable strip.

9. The apparatus of claim 1, wherein the stretchable strip is non-uniform in geometry along a length of the stretchable strip.

10. The apparatus of claim 1, wherein the stretchable strip is continuous along a length of the stretchable strip.

11. The apparatus of claim 1, wherein the stretchable strip is non-continuous along a length of the stretchable strip.

12. A system comprising:
a therapy tape comprising:
a strip of flexible material having a length and a width;
a second strip of flexible material disposed on the first strip of flexible material; and
a stretchable strip disposed on the second strip of flexible material, wherein the stretchable strip:
protrudes from a plane of the first side of the second strip of flexible material;
extends predominantly along the length of the second strip of flexible material; and
has a width, in at least one cross-section, that is less than the width of the first strip of flexible material; and
a backing strip applied to the first and second strips of flexible material of the therapy tape, the backing strip having a backing length and a backing width sufficient to cover the stretchable strip.

13. The system of claim 12, wherein the backing strip applied to the therapy tape is paper-based.

14. The system of claim 12, wherein the backing strip applied to the therapy tape comprises a first perforation across the backing width of the backing strip.

15. The system of claim 12, wherein the backing strip applied to the therapy tape comprises at least a first backing portion and a second backing portion, wherein the first backing portion extends along a length of the second strip of flexible material, and the second backing portion extends from approximately an end of the first backing portion to an end of the first strip of flexible material.

16. A method comprising:
forming a strip of flexible material to have a length and a width, wherein the strip of flexible material has a two-way stretch parallel to the length of the strip of flexible material;
applying an adhesive to at least a portion of a first side of the strip of flexible material; and
disposing a stretchable strip on the first side of the strip of flexible material, wherein the stretchable strip:
protrudes from a plane of the first side of the strip of flexible material;
extends predominantly along the length of the strip of flexible material; and
has a width, in at least one cross-section, that is less than the width of the strip of flexible material.

17. The method of claim 16, wherein the strip of flexible material comprises at least two overlapping strips of flexible material, and disposing the stretchable strip on the first side of the strip of flexible material comprises disposing a silicone structure on the first side of the strip of flexible material.

18. The method of claim 16, wherein disposing the stretchable strip on the first side of the strip of flexible material comprises applying the stretchable strip to a middle of the strip of flexible material to form margin regions on the first side of the strip of flexible material, the margin regions extending along the stretchable strip on opposite sides of the stretchable strip.

19. The method of claim 16, further comprising forming a first attachment region at a first end of the strip of flexible material and a second attachment region at a second end of the strip of flexible material opposite the first attachment region.

20. The method of claim 16, wherein disposing the stretchable strip comprises disposing the stretchable strip an entirety of the length of the strip of flexible material.
